(19) United States
(12) Patent Application Publication
(10) Pub. No.: US 2024/0095590 A1
(43) Pub. Date: Mar. 21, 2024

(54) SAMPLE COLLECTION CALL TIME PREDICTION SYSTEM AND METHOD

(71) Applicant: HITACHI HIGH-TECH CORPORATION, Tokyo (JP)

(72) Inventors: Kenichi TAKAHASHI, Tokyo (JP); Masatsuna TASAKA, Tokyo (JP)

(21) Appl. No.: 18/275,571

(22) PCT Filed: Jan. 31, 2022
(86) PCT No.: PCT/JP2022/003656

§ 371 Date: Aug. 2, 2023

(30) Foreign Application Priority Data
Mar. 4, 2021 (JP) 2021-034477

(51) Int. Cl. G06N 20/00

(52) U.S. Cl. CPC G06N 20/00

(57) ABSTRACT

Provided are a sample collection call time prediction system and a sample collection call time prediction method, with which it is possible to improve the accuracy of predicting the time at which a patient is called for sample collection. This call time prediction system includes a first processor that predicts, by machine learning, the time at which a patient is called for sample collection, the prediction being made on the basis of at least one of reception time information indicating the reception time for a patient from whom a sample is to be collected, sample type information indicating the type of the sample to be collected from the patient, a reception number indicating the order of reception of the patient, inpatient/outpatient classification information indicating whether the patient is an inpatient or an outpatient, and the number of waiting patients waiting to be called for sample collection at the reception time.

### TECHNICAL FIELD

The present invention relates to a sample collection call time prediction system and method.

### BACKGROUND ART

The sample collection assistance system is a system that assists sample collection work by automating and centrally managing peripheral tasks of sample collection such as patient reception for sample collection, patient call for sample collection, preparation of sample containers in response to test requests, barcode attachment to sample containers, and sample collection completion management, particularly regarding blood collection work at medical institutions.

Usually, a number is assigned to a patient at the same time as the reception of sample collection, and the patient understands that their turn has come by receiving a call according to that number.

A typical sample collection assistance system does not provide the patient with information about when the call will be made at the time of reception. Therefore, unless the staff of the medical institution provides the patient with information about when they will be called, or the patient predicts when they will be called by their own experience, the patient has to wait in the waiting room without knowing when the call will be made.

In a non-crowded time zone, it takes about a few minutes to be called, but in a busy time zone in the morning, the patient may wait for 30 minutes or more in the waiting room for a call, which is a heavy burden. On the other hand, there is known a system that predicts and calculates the waiting time for blood collection (see, for example, PTL 1).

### CITATION LIST

### Patent Literature

PTL 1: JP4156813B

### SUMMARY OF INVENTION

### Technical Problem

With the technique disclosed in PTL 1, the accuracy of waiting time prediction decreases due to operational changes in sample collection tasks and the like.

An object of the present invention is to provide a call time prediction system and method capable of improving the accuracy of predicting the time when a patient will be called for sample collection.

### Solution to Problem

To achieve the above object, a call time prediction system that is an example of the present invention includes a first processor that predicts a time that a patient will be called for sample collection by machine learning based on at least one or more of the following: reception time information indicating a reception time of a patient from whom a sample is to be collected, sample type information indicating a type of the sample to be collected from the patient, a reception number indicating the order in which the patient is received, inpatient/outpatient classification information indicating whether the patient is an inpatient or outpatient, and the number of patients waiting for a sample collection call at the reception time.

### Advantageous Effects of Invention

According to the invention, the prediction accuracy of the time when a patient is called for sample collection can be improved. Problems, configurations, and effects other than those described above will be clarified by the following description of the embodiments.

### BRIEF DESCRIPTION OF DRAWINGS

FIG. 1 is a schematic diagram of a sample collection call time prediction system in which a machine learning model is intended to be periodically updated.

FIG. 2 is information for predicting call time for sample collection using the machine learning model.

FIG. 3 is information for constructing the machine learning model used together with FIG. 2.

FIG. 4 is a parameter setting screen for constructing the machine learning model.

FIG. 5 is a sample collection receipt slip issued to a patient at the time of sample collection reception.

FIG. 6 is a predicted time guidance monitor screen displaying a list of patient call waiting states after reception of sample collection.

FIG. 7 is a schematic diagram of the sample collection call time prediction system in which the machine learning model is not periodically updated.

FIG. 8 is a schematic diagram of the sample collection call time prediction system having an entry management function of a sample collection waiting room.

### DESCRIPTION OF EMBODIMENTS

The configuration and operation of the call time prediction system according to the first to third embodiments of the present invention will be described below with reference to the drawings. One of the problems to be solved by the present embodiment is to allow the patient to effectively utilize the waiting time from reception of sample collection to call. In the embodiment, by presenting a predicted time of when a call will be made at the reception of sample collection, the patient is prompted to make effective use of the waiting time. Another problem to be solved by the embodiment is avoidance of congestion in the sample collection waiting room. By presenting the predicted call time for sample collection in advance, the patient is guided to gather at the sample collection place when the call time approaches. It contributes to social distancing, which is required to avoid infection with the recent novel coronavirus infection, and reduces the risk of infection for medical workers and patients.

### First Embodiment

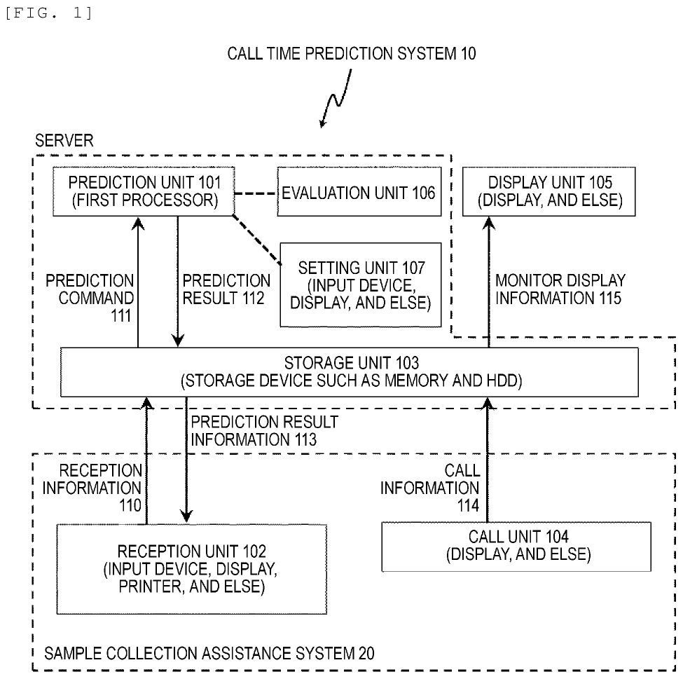

An embodiment of the present invention will be described below with reference to FIG. 1.

FIG. 1 is an overall schematic diagram of a sample collection call time prediction system 10, which is a first embodiment of the present invention, and is intended to periodically update a machine learning model.

The call time prediction system 10 includes a reception unit 102 that receives a patient for sample collection, a call unit 104 that informs the patient that it is the patient's turn to collect a sample, a storage unit 103 that stores information on reception and call of the patient, a prediction unit 101 that predicts the time from reception of sample collection until call by machine learning, and a display unit 105 that displays the prediction result on a monitor (for example, displayed by a browser).

The sample collection call time prediction system 10 of the present invention is assumed to cooperate with a sample collection assistance system 20 or be a part thereof to present the patient with the predicted call time for sample collection.

When cooperating with the sample collection assistance system 20, the reception unit 102 and the call unit 104 belong to the sample collection assistance system 20 side, and the storage unit 103 and the prediction unit 101 belong to the call time prediction system 10 side. The sample collection assistance system 20 and the call time prediction system 10 are connected via a local area network (LAN) in the medical facility or the Internet, and exchange information with each other. The display unit 105 belongs to the call time prediction system 10 side, but is assumed to be installed near the reception unit 102 and the call unit 104, and data is obtained from the storage unit 103 via the LAN in the medical facility or the Internet.

The patient who has received the sample collection instruction moves to the sample collection place, operates the provided reception unit 102, and registers themselves in the sample collection queue. Depending on the medical facility, taking out a medical chart may be linked to reception of sample collection. Here, the reception unit 102 automatically registers the patient in the waiting queue for sample collection upon receiving an instruction from the medical chart system.

In conjunction with the registration in the queue, the reception unit 102 transmits reception information 110 to the storage unit 103 to notify that the patient has been received.

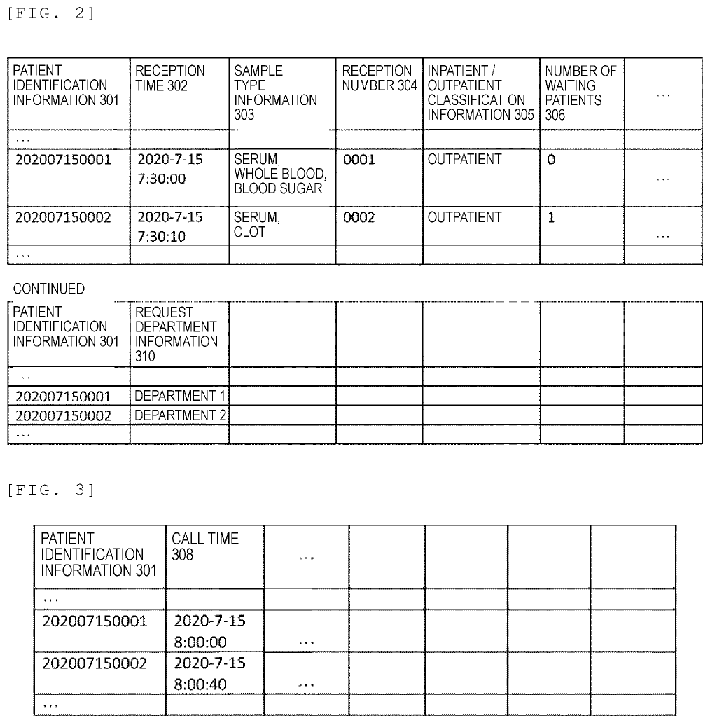

FIG. 2 illustrates a list of data related to the reception unit 102 obtained by the storage unit 103 through the reception information 110. The reception information 110 includes at least patient identification information 301 for identifying the patient who received the sample collection reception.

The patient identification information 301 is desirably a double key using patient ID or reception number and date. It is necessary to be able to link with the data of the same patient later received from the call unit 104, and to be able to distinguish the data of the same patient from the data of other days.

As information that the storage unit 103 must obtain without fail, there is a reception time 302 for sample collection. The reception time 302 of sample collection includes information on the year, month, day, hour, minute, and second when the reception unit 102 received the patient. Information on days of the week and holidays may be acquired from the information and used. It is also effective to express 24 hours as a real number, such as 13.5 for 1:30:00 PM, to indicate the time of the day.

As the reception time 302, the time of the reception unit 102, which is the information transmission side, may be provided in the reception information 110, or alternatively, the time of the storage unit 103, which is the reception side, may be used. If there are a plurality of reception units 102, it is conceivable that the respective clocks are out of sync, thus the internal clock of the reception side is preferably used in a uniform manner to avoid any confusion in the order.

When the reception unit 102 receives sample collection, if the data obtained and stored by the storage unit 103 include a part or all of sample type information 303 of a sample scheduled to be collected from the patient, a reception number 304 indicating the order of reception of the patient on the system, classification information 305 for distinguishing whether the patient is outpatient or inpatient, the number of waiting patients 306 for sample collection call at the time of reception of the patient, request department information 310 that instructed the sample collection to the patient, in addition to the patient identification information 301 and the reception time 302, the prediction accuracy of the call time of the prediction unit 101 is further improved. Such data do not necessarily need to be provided directly via the reception information 110, and may be provided or referenced from another location (server, system, and the like) using the patient identification information 301, for example.

The sample type information 303 is the type information of the sample to be collected. By referring to the sample type information 303, it is possible to determine, for example, which of the serum, whole blood, and plasma samples is to be collected, or how many types of samples are to be collected.

The reception number 304 is information on the number of receptions on the day of sample collection on the side of the clinical laboratory information system. If the information cannot be obtained, or if the number cannot be unified among a plurality of systems, the storage unit 103 side may internally count the number of the day and store and use the counted number as the reception number 304.

The classification information 305 is one of patient attribute information and indicates whether the patient who received the sample collection was an inpatient or an outpatient. It is effective when there is a difference in call patterns between inpatients and outpatients.

The number of waiting patients 306 indicates how many patients were waiting before the patient who received the sample collection reception. If the information cannot be obtained, the storage unit 103 side may internally count the number and store and use the counted number. If the exact number of patients cannot be obtained, even if the number of patients with an error of about 10% or about 10 is used instead, sufficient prediction accuracy can be ensured, so even if there is a slight difference, that numerical value is used as the number of waiting patients 306.

The request department information 310 is information as to which department within the medical institution instructed the patient to collect the sample. The information can be expected to be effective when there are differences in call patterns among departments, such as earlier calls depending on the requesting department.

After receiving the reception information 110, the storage unit 103 further issues a prediction command 111 to the prediction unit 101.

The prediction unit 101 can refer to the information received in the reception information 110 and the information processed based on the received information as they are, and predicts the call time for sample collection using an already built machine learning model. The prediction unit 101 provides a prediction result 112 to the storage unit 103 and the prediction result 112 is stored in the storage unit 103. The reason why the prediction result 112 is stored in the storage unit 103 is to evaluate how much the prediction result deviates from the actual call time, and to improve the prediction later.

The storage unit 103 uses prediction result information 113 to return the call time prediction result to the reception unit 102. The time of the prediction result 112 is represented by a real number, and the time of the prediction result information 113 is represented such as 1:30:00 PM.

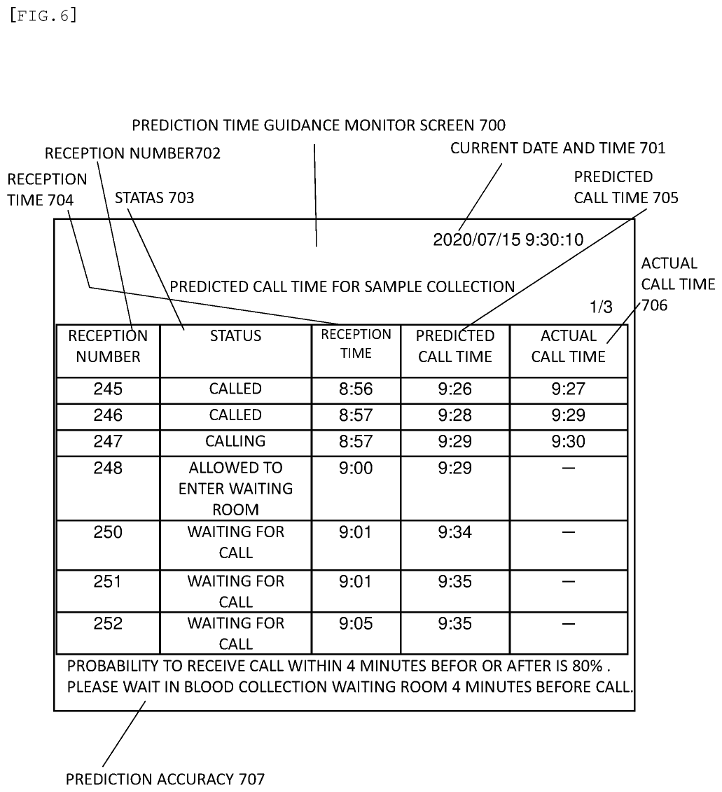

The reception unit 102 prints a reception date and time 601, a patient name 602, and a reception number 603, as well as a predicted call time 604 for sample collection, on a sample collection receipt slip 600 of FIG. 5, for notifying the patient that the reception is completed successfully, to inform the predicted call time. When printing the predicted call time 604, a prediction accuracy 605 is printed based on the past situation. For example, by writing the prediction accuracy 605 together, such as “the probability that the error between the predicted call time and the actual call time is within ±4 minutes is 80%”, the patient can understand how accurate the prediction is.

Based on the predicted call time 604 information, the patient can temporarily leave the sample collection waiting room, complete other tasks, and come back when the time comes. It can be expected that the patient's waiting time will be effectively used and the patient's stress will be reduced. By encouraging more patients to leave the waiting room, it is expected that it will contribute to social distancing, which is a common issue in recent novel coronavirus infections, and reduce the risk of infection between patients.

When it is the patient's turn who has completed the sample collection reception at the reception unit 102, the call unit 104 displays the patient's reception number on the monitor and prompts the patient to move to the sample collection place. Simultaneously, the call unit 104 transmits call information 114 to the storage unit 103 to notify that the patient has been called.

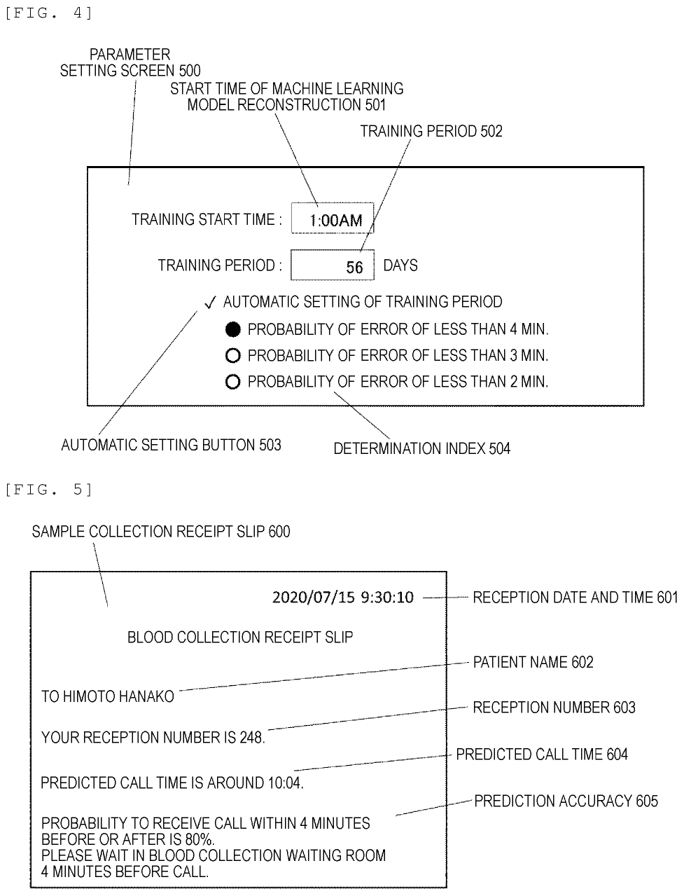

The call information 114 includes at least the patient identification information 301 for identifying the called patient. Using the patient identification information 301 as a key, it is possible to link with the reception information 110 to calculate the feature amount necessary for constructing the necessary machine learning model of the prediction unit 101 and to store the information in the storage unit 103 together. Simultaneously, among the information that the storage unit 103 must obtain is a call time 308 (FIG. 3). The call time 308 is based on the time, year, month, day, hour, minute, and second when the patient was called for sample collection. However, due to the specifications of the call unit 104, there may be times when the patient arrives at the sample collection place after being called. Here, the prediction accuracy is slightly reduced, but the prediction is sufficiently accurate to be used for prediction purposes, so the call time 308 may be used as it is in the prediction.

The call time 308 may provide the call information 114 with the time of the call unit 104, which is the transmission side of the information, or alternatively, the time of the storage unit 103, which is the reception side, may be used. Since it is conceivable that the clocks of the reception unit 102 and the call unit 104 are out of sync, the internal clock of the reception side is preferably used to avoid any confusion in the order.

An evaluation unit 106 constructs and updates the machine learning model using the information stored in the storage unit 103 when it is the time specified in a start time of machine learning model reconstruction 501 in a parameter setting screen 500 illustrated in FIG. 4, which belongs to a setting unit 107. Normally, it is desirable to set the time zone in the late-night hours after the end of the sample collection work.

In constructing the machine learning model, among the data stored in the storage unit 103, the data of the period specified as a training period 502 from the day before the prediction target date (today) is used as training data. Normally, it is specified in the range of about 14 to 84 days. The longer the period, the better the prediction accuracy tends to be. However, if the sample collection call pattern changed during the period due to operational changes in sample collection tasks, the period until the prediction follows the change becomes longer and the prediction accuracy during that time deteriorates. Conversely, the shorter the period, the lower the prediction accuracy and the shorter the period until the prediction result follows the change in call pattern.

The setting of the training period 502 can be automated by an automatic setting button 503 being checked and enabled. The evaluation unit 106 evaluates the call time predicted by the prediction unit 101 by the method designated as a determination index 504. For example, if the “probability that the error is within 4 minutes” is specified, the ratio of the total number of patients whose error is within ±4 minutes from the difference between the predicted call time and the actual call time is calculated, and it is determined that the higher the ratio, the better, that is, the higher the prediction accuracy. A machine learning model is built by automatically changing the training period 502 by 7 days, 14 days, . . . 84 days in increments of 7 days, and on the final day, the setting where the index “the probability that the error between the predicted call time and the actual call time is within +4 minutes” is the most excellent is automatically set as the training period 502. After that, the machine learning model used by the prediction unit 101 is rebuilt and used for prediction from the next day onward.

Normally, if a medical institution reviews sample collection tasks to improve the tasks, and calls become earlier on a certain day, the prediction accuracy is temporarily reduced immediately after the task review because the data before the task review is used for training. However, when the system is introduced, the training period 502 is automatically shortened immediately after the task review, and by training with the data ratio after the task review as high as possible, it can be expected that the period of deterioration will be shortened. When a certain amount of time has passed after the review, the training period 502 is automatically lengthened, and an effect of improving the prediction accuracy can be expected.

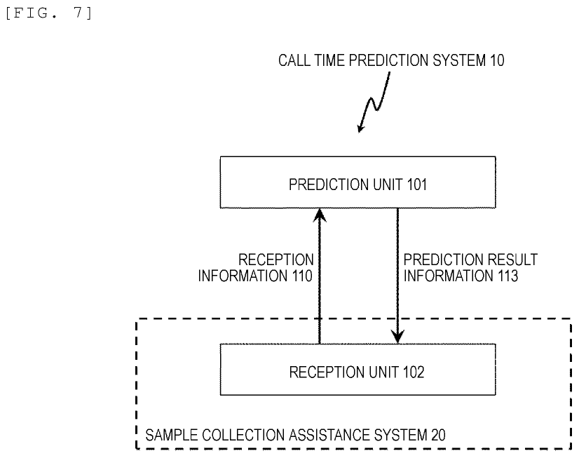

The display unit 105 is a terminal (personal computer (PC), mobile terminal, cellular phone, and the like) equipped with a display that displays a monitor screen to inform the call status of patients waiting for sample collection shown in a prediction time guidance monitor screen 700 in FIG. 6 to the patients in the waiting room or the like. On the monitor screen, a current date and time 701, a reception number 702 of the patient waiting for sample collection, a call status 703 of each reception number, a reception time 704 of each reception number, a predicted call time 705 of each reception number, an actual call time 706 of each reception number, and a call prediction accuracy 707, which are obtained through monitor display information 115 are displayed, and the information is updated as appropriate.

The current date and time 701 indicates what time it is now, and is useful for patients waiting for a call to understand how many minutes later they will be called in comparison with their own predicted call time 705.

The reception number 702 indicates which patient's information is the information in a horizontal row.

The status 703 displays the status of the call. The notation is changed to inform the status, such as “waiting for call” if sample collection has been received but has not yet been called; “called” if sample collection has already been called; and “calling” if it is right after the call of sample collection. It is convenient to set “calling” and “called” to be switched in the display automatically after a certain period of time, such as one minute later, but if information is obtained the sample collection has started, the state 703 may be switched from “calling” to “called” based on the information. The character color and background color of the notation may be changed according to the status 703. It is also possible to devise an easy-to-understand method by using symbols and figures instead of character strings.

The reception time 704 displays the reception time of sample collection for each reception number. It may be possible to select whether to display only hours and minutes or to display including hours, minutes, and seconds.

The predicted call time 705 displays the predicted call time for sample collection for each reception number. It may be possible to select whether to display only hours and minutes or to display including hours, minutes, and seconds. The remaining time may be indicated as how many minutes or how many seconds it takes to be called.

The actual call time 706 is displayed when a call is made to the patient waiting for sample collection. It may be possible to select whether to display only hours and minutes or to display including hours, minutes, and seconds. The information is useful information for patients called after the called patient, rather than the called patient themselves. It is possible to know and refer to the extent to which patients called before themselves are called late or early relative to the predicted call time 705 of the call time prediction system 10.

The prediction accuracy 707 is the accuracy of the predicted call time 705 obtained from past situations. For example, the prediction is presented to the patient waiting for a call to show the accuracy of the prediction, such as “the probability that the error between the predicted call time and the actual call time is within ±4 minutes is 80%” and ask for the understanding that there is a possibility of deviation. For the prediction accuracy 707 displayed here, the value specified as the determination index 504 on the parameter setting screen 500 (for example, an error of 4 minutes) and the probability calculated by the evaluation unit 106 (for example, 80%) can be used.

The features of the embodiment can also be summarized as follows.

As illustrated in FIG. 1, a call time prediction system 10 includes at least a first processor (prediction unit 101). The first processor (prediction unit 101) predicts the time that the patient will be called for sample collection by machine learning (artificial intelligence) based on at least one or more of the following: reception time information (reception time 302) indicating the reception time of a patient from whom a sample is to be collected, sample type information 303 indicating the type of the sample to be collected from the patient, a reception number 304 indicating the order in which the patient is received, inpatient/outpatient classification information 305 indicating whether the patient is an inpatient or outpatient, and the number of patients waiting for a sample collection call (number of waiting patients 306) at the reception time 302. Therefore, the patient can effectively utilize the waiting time until sample collection.

Specifically, as illustrated in FIGS. 2 and 3, the call time prediction system 10 includes a storage unit 103 (FIG. 1) that stores at least one or more of the reception time information (reception time 302), the sample type information 303, the reception number 304, the inpatient/outpatient classification information 305, the number of patients waiting for call (number of waiting patients 306), and measured value (call time 308) of time when the patient is called. Based on the data stored in the storage unit 103, the first processor (prediction unit 101) performs machine learning to predict the time when the patient will be called for sample collection. Therefore, a machine learning model can be rebuilt by comparing predicted value and the measured value of the time when a patient will be called for sample collection. The storage unit 103 is configured by a storage device such as a memory or a hard disk drive (HDD).

Specifically, the first processor (evaluation unit 106, FIG. 1) changes a training period 502 of the machine learning (FIG. 4) a plurality of times, constructs a provisional machine learning model for each training period, and determines the training period of the provisional machine learning model with the highest probability that the difference between the predicted value and the measured value of the time at which a patient is called is within a threshold value, which is an index of prediction accuracy (determination index 504, FIG. 4). After setting the determined training period, the first processor (evaluation unit 106, FIG. 1) reconstructs the machine learning model used in the task. As a result, the training period for machine learning can be automatically set, and the machine learning model can be rebuilt.

The call time prediction system 10 includes an output device (printer of a reception unit 102, display of a display unit 105, FIG. 1). The first processor (evaluation unit 106, FIG. 1) calculates a prediction accuracy that indicates the probability that the difference between the predicted value and the measured value of the time at which the patient will be called is within a threshold value. The output device outputs a predicted value of the time when the patient will be called (predicted call time 604 in FIG. 5 and predicted call time 705 in FIG. 6) and a threshold value that is an index of prediction accuracy (4 minutes of prediction accuracy 605 in FIGS. 5, and 4 minutes of prediction accuracy 707 in FIG. 6) and the prediction accuracy (80% of prediction accuracy 605 in FIG. 5 and 80% of prediction accuracy 707 in FIG. 6).

Therefore, the patient can check the predicted value of the time to be called for sample collection and the prediction accuracy. In the embodiment, the output device is a printer or a display. Therefore, the patient can visually check the predicted value of the time to be called and the prediction accuracy.

In the embodiment, the first processor (prediction unit 101, FIG. 1) predicts the time when the patient is called for sample collection by machine learning based on data in the storage unit 103 including at least the number of patients waiting for a call (number of waiting patients 306, FIG. 2). Specifically, the first processor (prediction unit 101, FIG. 1) predicts the time when the patient is called for sample collection by machine learning based on data in the storage unit 103 including at least the number of patients waiting for a call (number of waiting patients 306, FIG. 2) and the reception time information (reception time 302, FIG. 2). More specifically, the first processor (prediction unit 101, FIG. 1) predicts the time when the patient is called for sample collection by machine learning based on data in the storage unit 103 including at least the number of patients waiting for a call (number of waiting patients 306, FIG. 2), the reception time information (reception time 302, FIG. 2), and the reception number 304. According to the findings of the present inventors, the number of patients waiting for a call (number of waiting patients 306), the reception time information (reception time 302), and the reception number 304 have a large influence on prediction accuracy in this order.

The call time prediction system 10 includes a setting unit 107 (FIG. 1) that sets a training period for machine learning. The setting unit 107 includes, for example, an input device (keyboard, mouse, and the like) and a display. The first processor displays a parameter setting screen 500 (FIG. 4) on the display and receives an input value for each input item on the parameter setting screen 500 via the input device. It is possible to easily set the training period for machine learning.

The call time prediction system 10 includes the reception unit 102 (FIG. 1) that receives patients from whom samples are collected. The reception unit 102 is composed of, for example, an input device (such as a touch sensor of a touch panel), a display (such as a display of a touch panel), and a printer for printing a sample collection receipt slip 600. Therefore, the patient can complete the reception of sample collection by themselves.

The call time prediction system 10 includes a call unit 104 (FIG. 1) that calls the patients from whom samples are collected. The call unit 104 is composed of, for example, a display. Therefore, the patient can be notified that it is their turn to collect the sample.

As described above, according to the embodiment, it is possible to improve the accuracy of predicting the time at which a patient is called for sample collection.

### Second Embodiment

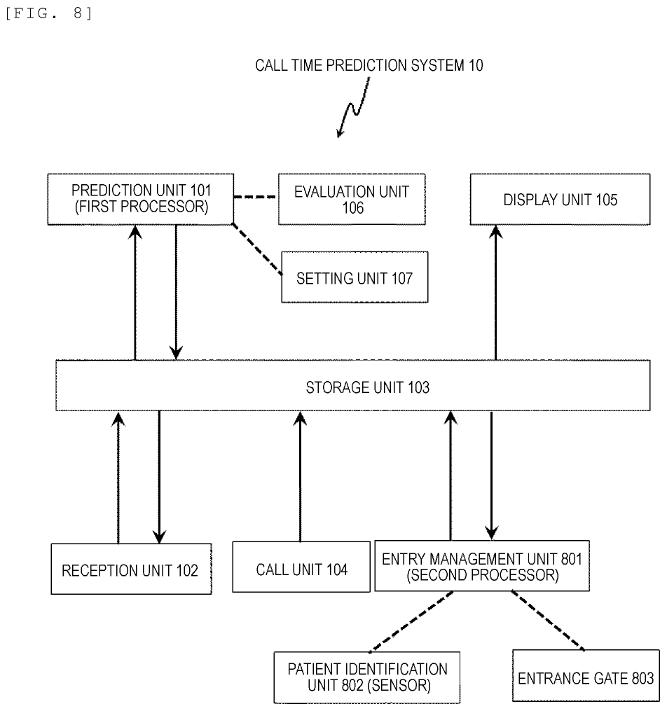

Another embodiment of the present invention will be described below with reference to FIG. 7.

FIG. 7 is an overall schematic diagram of the sample collection call time prediction system 10, which is a second embodiment of the present invention, and is intended to continue using a machine learning model once built.

The call time prediction system 10 is configured by the reception unit 102 that receives a patient for sample collection, and the prediction unit 101 that predicts the time from reception of sample collection until call by machine learning.

The sample collection call time prediction system 10 of the present invention is assumed to cooperate with the sample collection assistance system 20 or be a part thereof to present the patient with the predicted call time for sample collection.

When cooperating with the sample collection assistance system 20, the reception unit 102 is located on the sample collection assistance system 20 side, and the prediction unit 101 is located on the call time prediction system 10 side. The sample collection assistance system 20 and the call time prediction system 10 are connected via the LAN in the medical facility or the Internet, and exchange information with each other.

The patient who has received the sample collection instruction moves to the sample collection place, operates the provided reception unit 102, and registers themselves in the sample collection queue. Depending on the medical facility, taking out a medical chart may be linked to reception of sample collection. Here, the reception unit 102 automatically registers the patient in the waiting queue for sample collection upon receiving an instruction from the medical chart system.

In conjunction with registration in the queue, the reception unit 102 transmits the reception information 110 to the prediction unit 101 to provide information related to the received patient.

As illustrated in FIG. 2, the prediction unit 101 obtains data related to the reception unit 102 through the reception information 110. The reception information 110 includes at least the patient identification information 301 for identifying the patient who received the sample collection reception.

The patient identification information 301 is desirably a double key using patient ID and date or reception number and date.

As information that the prediction unit 101 must obtain without fail, there is a reception time 302 for sample collection. The reception time 302 of sample collection includes information on the year, month, day, hour, minute, and second when the reception unit 102 received the patient. Information on days of the week and holidays may be acquired from the information and used. It is also effective to express 24 hours as a real number, such as 13.5 for 1:30:00 PM, to indicate the time of the day. As the reception time 302, the time of the reception unit 102, which is the information transmission side, may be provided in the reception information 110, or alternatively, the time of the prediction unit 101, which is the reception side, may be used. If there are a plurality of reception units 102, it is conceivable that the respective clocks are out of sync, the internal clock of the reception side is preferably used to avoid any confusion in the order.

When the reception unit 102 receives sample collection, if the data obtained by the prediction unit 101 include a part or all of sample type information 303 of a sample to be collected from the patient, a reception number 304 indicating the order of reception of the patient on the system, classification information 305 for distinguishing whether the patient is outpatient or inpatient, the number of waiting patients 306 for a sample collection call at the time of reception of the patient, request department information 310 that instructed the sample collection to the patient, in addition to the patient identification information 301 and the reception time 302, the prediction accuracy of the call time of the prediction unit 101 is further improved. The data do not necessarily need to be provided directly via the reception information 110, and may be provided or referenced from another location using the patient identification information 301, for example.

The sample type information 303 is the type information of the sample to be collected. By referring to the sample type information 303, it is possible to determine which sample is to be collected, for example, a serum sample, a whole blood sample, or a plasma sample.

The reception number 304 is information on the number of receptions on the day of sample collection on the side of the clinical laboratory information system. If the information cannot be obtained, or if the number cannot be unified among a plurality of systems, the prediction unit 101 side may internally count the number of the day and store and use the counted number as the reception number 304.

The classification information 305 is one of patient attribute information and indicates whether the patient who received the sample collection was an inpatient or an outpatient. It is effective when there is a difference in call patterns between inpatients and outpatients.

The number of waiting patients 306 indicates how many patients were waiting before the patient who received the sample collection reception. If the information cannot be obtained, the storage unit 103 side may internally count the number and store and use the counted number. If the exact number of patients cannot be obtained, even if the number of patients with an error of about 10% or about 10 is used instead, sufficient prediction accuracy can be ensured, so even if there is a slight difference, that numerical value is used as the number of waiting patients 306.

The request department information 310 is information as to which department within the medical institution instructed the patient to collect the sample. The information can be expected to be effective when there are differences in call patterns among departments, such as earlier calls depending on the requesting department.

The prediction unit 101 uses the information received in the reception information 110 and the information processed based on the received information as they are, and predicts the call time for sample collection using an already built machine learning model. The prediction result is transmitted to the reception unit 102 via the prediction result information 113.

The reception unit 102 prints the reception date and time 601, the patient name 602, and the reception number 603, as well as the predicted call time 604 for sample collection, on the sample collection receipt slip 600 of FIG. 5, for notifying the patient that the reception is completed successfully, to inform the predicted call time. When printing the predicted call time, the prediction accuracy 605 is printed based on the past situation. For example, by writing the prediction accuracy together, such as “the probability that the error between the predicted call time and the actual call time is within ±4 minutes is 80%”, the patient can understand how accurate the prediction is.

Based on the call time prediction information, the patient can temporarily leave the sample collection waiting room, complete other tasks, and come back when the time comes. It can be expected that the patient's waiting time will be effectively used and the patient's stress will be reduced. By encouraging more patients to leave the waiting room, it is expected that it will contribute to social distancing, which is a common issue in recent novel coronavirus infections, and reduce the risk of infection between patients.

### Third Embodiment

Another embodiment of the present invention will be described below with reference to FIG. 8.

FIG. 8 is an example in which the sample collection call time prediction system 10 of FIG. 1, which is the third embodiment of the present invention, is applied to management of the number of entering and exiting patients in the sample collection waiting room.

The reception unit 102 is installed outside the sample collection waiting room. A patient who has completed the reception for sample collection receives the sample collection receipt slip 600 in FIG. 5 and checks what time their sample collection call will come and around what time they should come to the sample collection room.

An entrance gate 803 is provided at the entrance of the sample collection waiting room, and only patients who meet certain conditions and their attendants can enter the room. Therefore, a patient who receives a sample collection reception at the reception unit 102 spends time in a place other than the blood collection waiting room after the reception.

A patient identification unit 802 is installed in front of the entrance gate 803. If a patient identifier such as a reception number or a patient ID is printed on the sample collection receipt slip 600 in barcode form, the patient identification unit 802 can automatically identify the patient using a barcode reader.

An entry management unit 801 accesses the storage unit 103 using the patient identification information 301 obtained from the patient identification unit 802 as a key, and obtains the predicted sample collection call time of the patient. If it is within a certain time of the predicted call time, for example, 4 minutes before the predicted call time, entry is permitted, the entrance gate 803 is opened, and the patient is urged to enter the blood collection waiting room. If it is not within a certain time (here, 4 minutes before the predicted call time), the user is prompted to try entering the room again after the time has come.

As illustrated in FIG. 6, by displaying that it is possible to enter the waiting room, such as “allowed to enter the waiting room” in the column of the status 703 of the reception patient of the predicted time guidance monitor screen 700, it will be possible to indicate which patients can enter the waiting room. Here, the guidance monitor screen 700 is preferably installed outside the waiting room.

The features of the embodiment can also be summarized as follows.

As illustrated in FIG. 8, the call time prediction system 10 includes a sensor (patient identification unit 802) that detects patient identification information 301 indicating information identifying a patient, a gate (entrance gate 803) installed in the waiting room, and a second processor (entry management unit 801). The second processor (entry management unit 801) determines whether the patient can enter the waiting room based on the predicted value of time when the patient corresponding to patient identification information 301 is called and a current time, and if it is determined that the room can be entered, the gate is opened, and if it is determined that the room cannot be entered, the gate (entrance gate 803) is controlled to be closed. As a result, congestion in the waiting room can be reduced.

With the above method, by limiting the entry of patients waiting for sample collection into the waiting room to those within a certain period of time from the predicted call time and their attendants, congestion in the waiting room is alleviated and the risk of the infection with the current novel coronavirus can be reduced.

The present invention is not limited to the above-described embodiments, and includes various modifications. For example, the embodiments described above are those described in detail to describe the present invention in an easy-to-understand manner, not necessarily limited to those having all the configurations described. It is possible to replace a part of the configuration of one embodiment with the configuration of another embodiment, and it is also possible to add the configuration of the other embodiment to the configuration of one embodiment. It is possible to add, delete, or replace another configuration for a part of the configuration of each embodiment.

Each of the above configurations, functions, and the like may be implemented by hardware, for example, by designing a part or all of them using an integrated circuit. Each of the above configurations, functions, and the like may be implemented by software by a processor interpreting and executing a program for implementing each function. Information such as programs, tables, and files that implement each function can be stored in a recording device such as memory, hard disk, solid state drive (SSD), or a recording medium such as IC card, SD card, and DVD.

For example, the prediction unit 101 or the entry management unit 801 may be configured with an integrated circuit. Such configuration improves the processing speed compared to when a processor executes software to perform processing. In the third embodiment, the second processor (entry management unit 801) is separate from the first processor (prediction unit 101), but the processors may be configured integrally.

In the above embodiment, the prediction unit 101, the storage unit 103, the evaluation unit 106, and the setting unit 107 are implemented as functions of one server (FIG. 1), but may be implemented as respective functions of a plurality of servers.

The embodiment of the present invention may be the following aspects.

(1). A sample collection call time prediction system 10, including: a reception unit 102 for patients waiting for sample collection; and a prediction unit 101 for predicting the time for a call for sample collection by machine learning, in which the prediction unit 101 predicts the time when a patient is called for sample collection based on at least one or more of reception time information (reception time 302) when sample collection was received for the patient, sample type information 303 of a sample to be collected from the patient, reception number 304 indicating the reception order of the patient on the system, inpatient/outpatient classification information 305 that distinguishes whether the patient is an inpatient or an outpatient, and the number of patients waiting for a sample collection call at the time of reception of the patient (number of waiting patients 306).

(2). The sample collection call time prediction system 10 according to (1), further including: a call unit 104 for calling a sample collection patient; and a storage unit 103 for storing information related to the patient, in which the storage unit 103 stores at least one or more of the reception time information (reception time 302) when sample collection was received for the patient, the sample type information 303 of the sample to be collected from the patient, the reception number 304 indicating the reception order of the patient on the system, the inpatient/outpatient classification information 305 that distinguishes whether the patient is an inpatient or an outpatient, the number of patients waiting for a sample collection call at the time of reception of the patient (number of waiting patients 306), and call time information (call time 308) when the patient is called, and the prediction unit 101 is trained to predict the patient call time based on the information stored in the storage unit 103.

(3). The sample collection call time prediction system according to (2), further including: a setting unit 107 that defines (sets) a period (training period 502) of training data used for constructing a learning model; and an evaluation unit 106 that compares a call time prediction result and an actual measurement time and evaluates the prediction accuracy, in which before the prediction unit 101 reconstructs the learning model, the evaluation unit 106 automatically switches the training period to construct a provisional learning model, compares and evaluates the predicted call time value and the actual measurement result, and automatically sets a training period that improves the prediction accuracy in the setting unit 107, and the prediction unit 101 allows to automatically set a more appropriate training period in reconstructing the learning model.

(4). The sample collection call time prediction system 10 according to (2), further including: a setting unit 107 that defines (sets) an index of prediction accuracy (determination index 504); an evaluation unit 106 that calculates prediction accuracy using the defined index; and a display unit 105 that displays the call prediction status of a plurality of patients waiting for blood collection on a monitor, in which when the reception unit 102 or the display unit 105 presents the predicted call time for sample collection for the patients, the prediction accuracy index (determination index 504) defined in the setting unit 107 is presented together with a prediction accuracy 605 calculated in the evaluation unit 106.

(5). The sample collection call time prediction system 10 according to (2), further including: an entry management unit 801 that determines whether a patient who has received sample collection can enter the waiting room; a patient identification unit 802 that obtains patient identification information of the patient; and an entrance gate unit (entrance gate 803) disposed to separate the sample collection waiting room and other areas, in which the entry management unit 801 obtains the predicted sample collection time of the patient (predicted call time 705) from the storage unit 103 based on the patient identification information obtained from the patient identification unit 802, and determines whether the room can be entered based on the information, and the entrance gate unit (entrance gate 803) controls the entry and exit of people in the waiting room for sample collection by opening a gate when the entry is allowed, and closing the gate when entry is not allowed.

According to (1) to (5), it is possible to predict with higher accuracy the process from the sample collection reception to the sample collection call. As one of prediction methods that can be easily implemented, a method of using an average value for the same time zone as a prediction value based on the actual waiting time until a call in the past is conceivable. When using such method, in the evaluation, 13 to 67% of all samples had an error within ±3 minutes between the predicted and measured values. Accordingly, when predicted by the present invention, better results of 65 to 91% could be obtained.

### REFERENCE SIGNS LIST

10: call time prediction system
20: sample collection assistance system
101: prediction unit
102: reception unit
103: storage unit
104: call unit
105: display unit
106: evaluation unit
107: setting unit
110: reception information
111: prediction command
112: prediction result
113: prediction result information
114: call information
115: monitor display information
301: patient identification information
302: reception time
303: sample type information
304: reception number
305: inpatient/outpatient classification information
306: number of waiting patients
308: call time
310: request department information
500: parameter setting screen
501: start time of machine learning model reconstruction
502: training period
503: automatic setting button
504: determination index
600: sample collection receipt slip
601: reception date and time
602: patient name
603: reception number
604: predicted call time
605: prediction accuracy
700: prediction time guidance monitor screen
701: current date and time
702: reception number
703: status
704: reception time
705: predicted call time
706: actual call time
707: prediction accuracy
801: entry management unit
802: patient identification unit
803: entrance gate

1.-13. (canceled)

14. A sample collection call time prediction system comprising:
a storage unit for storing reception time information indicating a reception time of a patient from whom a sample is to be collected, sample type information indicating a type of the sample to be collected from the patient, a reception number indicating the order in which the patient is received, inpatient/outpatient classification information indicating whether the patient is an inpatient or an outpatient, a number of patients waiting for a sample collection call at the reception time, and a measured value of a time at which the patient is called; and
a first processor that predicts a time at which the patient will be called for sample collection by machine learning based on data stored in the storage unit, wherein
the first processor
starts reconstruction of a machine learning model at a specified time within a certain day and changes a training period at fixed intervals within a specified period to construct a provisional machine learning model for each training period,
determines the training period for the provisional machine learning model with the highest probability that a difference between the predicted value and the measured value of the time at which the patient is called is within a threshold value, which is an index of prediction accuracy, and
reconstructs the machine learning model used in the task, after the determined training period is set.

15. The sample collection call time prediction system according to claim 14, further comprising:
an output device, wherein
the first processor calculates the prediction accuracy indicating the probability that the difference between the predicted value and the measured value of the time at which the patient is called is within the threshold value, and
the output device outputs the predicted value of the time at which the patient will be called, the threshold value that is an index of the prediction accuracy, and the prediction accuracy.

16. The sample collection call time prediction system according to claim 14, further comprising:
a sensor for detecting patient identification information indicating information identifying the patient;
a gate installed in a waiting room; and
a second processor that determines whether the patient can enter the waiting room from the predicted value of the time at which the patient corresponding to the patient identification information is called and a current time, and controls the gate to open if it is determined that entry is allowed, and to close the gate if it is determined that entry is not allowed.

17. The sample collection call time prediction system according to claim 14, wherein
the first processor predicts the time at which the patient will be called for sample collection by machine learning based on the data including at least the number of patients waiting for the call.

18. The sample collection call time prediction system according to claim 17, wherein
the first processor predicts the time at which the patient will be called for sample collection by machine learning based on the data including at least the reception time information.

19. The sample collection call time prediction system according to claim 18, wherein
the first processor predicts the time at which the patient will be called for sample collection by machine learning based on the data including at least the reception number.

20. The sample collection call time prediction system according to claim 14, further comprising:
a reception unit that receives the patients from whom the sample is collected.

21. The sample collection call time prediction system according to claim 20, further comprising:
a call unit that calls the patient from whom the sample is collected.

22. The sample collection call time prediction system according to claim 14, further comprising:
a setting unit for setting the training period for the machine learning.

23. The sample collection call time prediction system according to claim 15, wherein
the output device is a printer or a display.

24. A sample collection call time prediction method for predicting a time at which a patient will be called for sample collection by machine learning based on reception time information indicating a reception time of a patient from whom a sample is to be collected, sample type information indicating a type of the sample to be collected from the patient, a reception number indicating the order in which the patient is received, inpatient/outpatient classification information indicating whether the patient is an inpatient or an outpatient, a number of patients waiting for a sample collection call at the reception time, and a measured value of the time at which the patient is called, the method comprising:
starting reconstruction of a machine learning model at a specified time within a certain day and changing a training period at fixed intervals within a specified period to construct a provisional machine learning model for each training period,
determining the training period for the provisional machine learning model with the highest probability that a difference between the predicted value and the measured value of the time at which the patient is called is within a threshold value, which is an index of prediction accuracy, and
reconstructing the machine learning model used in the task, after setting the determined training period.
